(19) United States
(12) Patent Application Publication
(10) Pub. No.: US 2007/0239195 A1
(43) Pub. Date: Oct. 11, 2007

(54) Adjustable Prosthetic Band

(76) Inventor: David Jean-Marie Nocca, Montpellier (FR)

Correspondence Address: WOLF GREENFIELD & SACKS, P.C., 600 ATLANTIC AVENUE, BOSTON, MA, 02210-2206, US

(21) Appl. No.: 11/597,033

(22) PCT Filed: May 17, 2005
(86) PCT No.: PCT/FR05/01229

§ 371 Date: Apr. 6, 2007

(30) Foreign Application Priority Data
May 18, 2004 (FR) 04 05404

(51) Int. Cl. A61M 29/00

(52) U.S. Cl. 606/191

(57) ABSTRACT

The invention concerns a band comprising several perpendicular graduations (7, 8) on the first large side (2) and spaced apart from one another along the direction parallel to the first large side, respectively first and second graduation holes (6) being associated with each graduation, and two first and second suturing holes (9) are arranged in a second section (1) of the band.

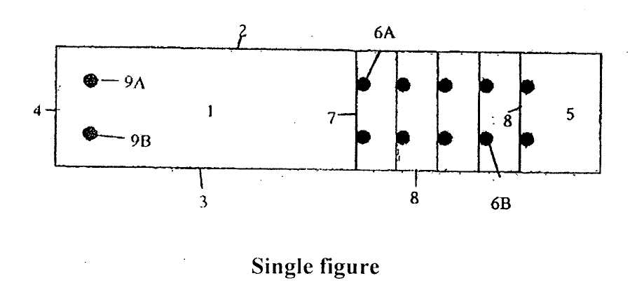

Surgery for morbid obesity, or bariatric surgery, has become a self-standing discipline within digestive surgery. The major reasons for this are the ineffectiveness in the long term of the various therapeutic approaches (diet, drugs, psychology), as well as the microinvasive aspect provided by laparoscopy techniques.

At present, several surgical procedures are used, working either by a restrictive mechanism (calibrated vertical gastroplasty, adjustable calibrated horizontal gastroplasty) or by a combination, that is, a restriction of the gastric capacity as well as a digestive intestinal shunt, bringing about a malabsorption (gastric shunting, duodenal inversion).

Certain technical problems are common to all these procedures. Erosion of the stomach wall by the various prosthetic materials put in place to attempt to calibrate the gastric pouches created to achieve a process of restriction is one of the major concerns of the surgeons.

This is not a new complication, since Dr. Angelchik had developed in the eighties a silicone prosthesis placed at the level of the cardia in order to combat gastroesophageal reflux. The efficacy was interesting, but migration of the prosthesis to an intragastric position were described in many cases (11%), leading surgeons to abandon this technique.

Other materials, such as MARLEX (polypropylene) have been used to calibrate the gastric pouch in CVG. The same phenomenon was noted by surgeons, with rates of intragastric migration of 24% being found in the series of MacLean 1993.

Fobi's use of silicone in the form of a ring made of “Silastic” (trademark) to calibrate the gastric shunt procedure in the area of the gastric pouch also has this drawback.

The siliconized adjustable gastric ring is the technique most often used today in Europe. Thus far, the erosion complication has been given little attention, but recent publications present disturbing findings with rates of erosion as high as 10%, along with serious consequences even leading to the death of the patient (blockage, bleeding).

There are various explanations as to the physiopathology of this complication:

Microtraumatisms of the stomach wall produced when putting the ring in place are mentioned and these should lead one to prefer making a dissection with the posterior portion of the stomach in view, rather than a blind dissection, which may produce an undiagnosed lesion.

An emplacement too close to the esophagus, which does not have a serous tunic, has also been suggested.

A chronic erosion by the rigid parts of the rings (especially the edge of the Dacron mesh of the SAGB) is mentioned by Zimmerman.

The invention concerns a band which mitigates the above drawbacks by reducing the risk of erosion and allowing the surgeon to make a precise adjustment during or soon after the operation.

This is accomplished by a flexible, nonextensible band, having a large side and an imaginary mean perpendicular of the first large side, made of a material which is resorbable longer than two years and colonizable by fibroblasts,

characterized in that, on the same surface and on a first piece of the band situated on a first side of the mean perpendicular of the band, there are made several graduation lines perpendicular to the first large side and at a distance from each other in the direction parallel to the first large side, two first and second graduation holes respectively being associated with each graduation line, and two first and second suture holes are made in a second piece of the band, situated on a second side of the mean perpendicular. By line is meant any visual reference feature.

Thanks to the choice of the not very rigid material and thanks to the possibility of adjusting the ring formed from the band by using the graduation lines, one obtains a band that presents slight risk of erosion and that is adjustable during and especially soon after the operation, by cutting the securing threads of the band, readjusting, and putting two new closure threads in place.

The material which is resorbable longer than two years and colonizable by fibroblasts is preferably collagen of animal origin,

especially of porcine origin, with a preference for a cellular dermal collagen containing elastin fibers. One can obtain a collagen band of this type under the brand “Pelvicol” from the applicant, 164 avenue J. Kessel portik P4 78960 Voisins le Bretonneux. This is an implant of porcine origin, consisting of a cellular dermal collagen containing elastin fibers. It is thus a heterograft in the form of a sheet imbued with physiological solution. This implant allows one to have a good quality of suture and it is colonized by fibroblasts and revascularized. One can also use an extracellular collagen matrix of porcine intestinal submucosa, known under the name Stratadis, which one can obtain from the Cook Le Levant laboratory at 2 rue du Nouveau Bercy 94227 Charenton.

Preferably, on the same surface of the band is made at least one first supplemental graduation line on the first piece at a first given distance from one of the first lines and parallel with the latter, and preferably on the same surface of the band, there are several first supplemental graduation lines on the first piece at the given distance from each other and parallel to the first line. One can thus modify soon after the operation the diameter of the ring formed by the band, if such is necessary. This maneuver is further facilitated by having on the same surface of the band a second supplemental graduation line on the second piece at a second given distance from one of the first lines and parallel with it. Preferably, the first and the second distances are equal, which facilitates the adjustment maneuvers.

To facilitate the placement of the band, two holes are provided tangent to each of the first graduation lines. The holes can have a diameter of 1 to 3 mm. These allow for the passage of a suture thread to close the band. Preferably, each hole on a first graduation line is aligned parallel with the large side with one hole tangent to the other first graduation line. Preferably, the holes are at a distance apart that is equal to their distance from the large sides.

The lines can be made with ink. By line is meant any visible reference feature.

According to one embodiment particularly adapted to the anatomy, the length of the main piece is 5 cm and the distance between the lines on the same piece is 1 cm. The band can have a width between 1.5 and 3 cm and preferably the width is 2 cm. It can have, in particular, a thickness of 0.5 to 5 mm.

The band is used as follows. The distal end of the gastric pouch is encircled by a band according to the invention and this is closed into a ring by passing suture threads into two superimposed holes. In this way, one performs a calibration of the gastric pouch of the calibrated vertical gastroplasty or any bariatric surgery procedure requiring a calibration. According to another possibility, one short-circuits the stomach by connecting the gastric pouch directly to a portion of the intestine and by placing a band according to the invention, curved into a ring, on this junction, preferably on the gastric pouch. A post-operative check to verify the proper quality of the calibration is done during the early post-operative period by esogastric opacification. If the outcome is not satisfactory, one can reoperate on the patient and adjust the diameter of the band by using the supplemental graduation lines still visible, and the band can be loosened and readjusted once again.

The single FIGURE of the enclosed drawing illustrates the invention.

The band shown in the FIGURE is made of Pelvicol. It consists of a second piece 1 with a length of 5 cm and a width of 2 cm. The piece 1 has two large sides 2, 3 and one small side 4. The band contains a first end piece 5 having a width of 2 cm and a length of 5 cm.

Two holes 6A, 6B are made on the piece 5, having a diameter of 2 mm and being tangent to a first graduation line 7, which is placed on one surface of the band perpendicular to the large sides 2, 3. The holes 6 are arranged at equal distance from the large sides 2, 3. The line 7 adjacent to the piece 1 is on the mean perpendicular of the large side 2.

On this same surface, which is destined to be on the distal exterior of the viscera once put in place, there are four supplemental graduation lines 8 on the piece 5, equally spaced by 1 cm from each other, the first of them being 1 cm from the first graduation line 7; having a thickness of 0.2 to 1 mm.

On the second piece 1 are punctured two suture holes 9A, 9B; the holes 9A and 6A, on the one hand, and 9B and 6B, on the other, are aligned respectively along two imaginary lines parallel to the large side 2.

Thanks to these lines 7 and holes 6, one can adapt the dimension of the prosthesis to the anatomical conditions of the dissection and to the size of the gastric pouch which is created. There are fewer erosion type complications and the cost of the operation is less.

1. A flexible, nonextensible band, having a large side and an imaginary mean perpendicular of the first large side, made of a material which is resorbable longer than two years and colonizable by fibroblasts, wherein, on a same surface and on a first piece of the band situated on a first side of the mean perpendicular of the band, there are several graduation lines perpendicular to the first large side and, at a distance from each other in a direction parallel to the first large side, two first and second graduation holes respectively being associated with each graduation line, and two first and second suture holes are made in a second piece of the band, situated on a second side of the mean perpendicular to the first large side.

2. The band as claimed in claim 1, wherein the first and second graduation holes associated with the same graduation line are at the same distance, the one from the first large side and the other from an opposite large side.

3. The band as claimed in claim 2 wherein the first graduation holes are aligned in a direction parallel to the first large side.

4. The band as claimed in claim 3, wherein the second graduation holes are aligned in a direction parallel to the first large side.

5. The band as claimed in claim 1, wherein the first suture hole is aligned with the first graduation holes (6A) in a direction parallel to the first large side.

6. The band as claimed in claim 5, wherein the second suture hole is aligned with the second graduation holes in a direction parallel to the first side.

7. The band as claimed in claim 1, wherein the graduation lines are equidistant.

8. The band as claimed in claim 1, wherein the graduation holes are tangent to their line.

9. The band as claimed in claim 1, wherein the distance between the first suture hole and the first graduation hole closest to it is 5 cm, and the distance between each graduation line is 1 cm.

10. The band as claimed in claim 1, wherein the several graduation lines comprises at least three graduation lines.

11. The band as claimed in claim 1, wherein the width of each graduation line is in a range between approximately 0.2 mm to approximately 1 mm.

12. The band as claimed in claim 1, wherein each graduation line extends substantially across the width of the band.

13. A method for treatment of obeseity, in which a distal end of a gastric pouch and an intestine is encirled by a band according to claim 1, wherein the band is closed into a ring by passing a suture thread through a graduation hole and a suture hole, that is superimposed on the graduation hole.

14. A flexible band, comprising:
a strip of material having a first edge long and a surface, the strip having an imaginary line extending perpendicular to the first long edge at approximately a middle of the strip to define a first section on one side of the middle and a second section on another side of the middle; and
a plurality of spaced-apart graduation lines extending substantially perpendicular to the first long edge and disposed on the surface in the second section.

15. The band as claimed in claim 14, further comprising a plurality of graduation holes, each hole associated with a respective graduation line.

16. The band as claimed in claim 15, wherein the plurality of graduation holes comprises a plurality of sets of graduation holes, each set of graduation holes comprising first and second graduation holes, each set of graduation holes being associated with a respective graduation line.

17. The band as claimed in claim 15, further comprising at least one suture hole disposed in the first section.

18. The band as claimed in claim 17, wherein the at least one suture hole comprises first and second suture holes.

19. The band as claimed in claim 14, wherein the strip is nonextensible.

20. The band as claimed in claim 14, wherein the strip is formed of a material that is adapted to be completely resorbed after two years.

21. The band as claimed in claim 14, wherein the strip is formed of a material that is adapted to be colonizable by fibroblasts.

22. The band as claimed in claim 15, wherein the graduation holes are aligned in a direction parallel to the first long edge.

23. The band as claimed in claim 17, wherein the suture hole is aligned with the graduation holes in a direction parallel to the first long edge.

24. The band as claimed in claim 15, wherein each graduation hole is arranged tangent to a corresponding graduation line.

25. The band as claimed in claim 17, wherein a distance between the suture hole and a first graduation hole that is disposed closest to the suture hole is approximately 5 cm.

26. The band as claimed in claim 14, wherein a distance between each graduation line is approximately 1 cm.

27. The band as claimed in claim 14, wherein the plurality of graduation lines comprises at least three graduation lines.

28. The band as claimed in claim 14, wherein a width of each graduation line is in a range between approximately 0.2 mm to approximately 1 mm.

29. The band as claimed in claim 14, wherein each graduation line extends substantially across a width of the strip.

30. The band as claimed in claim 14, wherein the strip is formed of a resorbable material.

31. A surgical method comprising:
providing a band formed of a strip of material having a first long edge and a surface, the strip having an imaginary line extending perpendicular to the first long edge at approximately a middle of the strip to define a first section on one side of the middle and a second section on another side of the middle and a plurality of spaced-apart graduation lines extending substantially perpendicular to a first long edge and disposed on the surface in the second section, each graduation line having a corresponding graduation hole, the band having a suture hole disposed in the first section;
encircling a gastro-intestinal organ with the band such that the suture hole aligns with one of the graduation holes; and
passing a suture through the graduation hole and the aligned suture hole.

32. The method as claimed in claim 31, wherein providing the band comprises providing a band formed of a strip of resorbable material.
